SEQUENCE LISTING (1 sequence)

SEQ ID NO: 1 - DNA, 19 bp, synthetic construct
  source 1..19: mol_type = other DNA
tatcttaagtcttcgcgtg

(19) United States
(12) Patent Application Publication
(10) Pub. No.: US 2025/0320569 A1
(43) Pub. Date: Oct. 16, 2025

(54) MASSIVELY MULTIPLEXED RAMAN OPTICAL BARCODING FOR ANALYTE DETECTION

(71) Applicant: The Trustees of Columbia University in the City of New York, New York, NY (US)

(72) Inventors: Zhilun ZHAO, New York, NY (US); Naixin QIAN, New York, NY (US); Wei MIN, New York, NY (US)

(21) Appl. No.: 19/202,549

(22) Filed: May 8, 2025

Related U.S. Application Data
(63) Continuation of application No. PCT/US2023/079101, filed on Nov. 8, 2023.
(60) Provisional application No. 63/424,223, filed on Nov. 10, 2022.

(51) Int. Cl. C12Q 1/70; C12Q 1/6825; G01N 21/65

(52) U.S. Cl. CPC C12Q 1/701; C12Q 1/6825; G01N 21/65; C12Q 2600/16; C12Q 2600/166; G01N 2021/655

(57) ABSTRACT

A Raman spectroscopy-based platform for massively multiplexed detection of analytes of interest, such as nucleic acids or peptides, and a hardware platform for economical and high-throughput detection.

### CROSS-REFERENCE TO RELATED APPLICATIONS

This application is a continuation of PCT International Application No. PCT/US2023/079101, filed Nov. 8, 2023, which claims benefit of U.S. Provisional Application No. 63/424,223, filed Nov. 10, 2022, the contents of each of which are hereby incorporated by reference.

### STATEMENT OF GOVERNMENTAL INTEREST

This invention was made with government support under grant numbers GM 128214 and GM 132860 awarded by the National Institutes of Health. The government has certain rights in the invention.

### REFERENCE TO SEQUENCE LISTING

This application incorporates-by-reference nucleotide sequences which are present in the file named “231109_91962-A -PCT_Sequence_Listing_AWG.xml”, which is 1,804 bytes in size, and which was created on Nov. 8, 2023 in the IBM-PC machine format, having an operating system compatibility with MS-Windows, which is contained in the XML file filed May 8, 2025 as part of this application.

### BACKGROUND

The disclosures of all publications, patents, patent application publications and books referred to in this application are hereby incorporated by reference in their entirety into the subject application to more fully describe the art to which the subject invention pertains.

Detecting and observing the subjects of interest is one of the fundamental approaches to studying biological processes. Y et the biological system is complicated in all aspects, and is challenging at the cellular and subcellular level. Therefore, there is an ongoing need for methods capable of detecting and analyzing a large number of biological molecules simultaneously and accurately.

### SUMMARY OF THE INVENTION

Herein a Raman spectroscopy-based platform for massively multiplexed detection of analytes of interest is disclosed, including massively multiplexed labeling assays, and a hardware platform for economical and high-throughput detection. Non-limiting examples of use of the platform in multiplex detecting nucleic acids or peptides/proteins are demonstrated.

According to certain exemplary embodiments of the present disclosure, a method is provided for detecting the presence of one or more different analytes present in a sample comprising:
(a) contacting the sample with a plurality of bead types, each bead type comprising:
   (1) one, or more, types of Raman-active small molecule(s) (RASM s), each at a predefined concentration in the bead, and
   (2) a binding molecule, affixed thereon, specific for one of each of said one or more different analytes;
   wherein each bead type differs from the remaining bead types of the plurality in (i) concentration of Raman-active small molecule(s), (ii) spectrum or spectra of Raman-active small molecule(s), or (ii) both concentration and spectrum/spectra pattern of Raman-active small molecule(s);
(b) removing unbound analytes or unbound beads; and
(c) determining, with Stimulated Raman Scattering (SRS) or Spontaneous Raman Scattering, which bead type(s) having an analyte bound thereto are present after step (b) so as to thereby determine which type(s) of analyte are bound,
thereby detecting which of one or more different analytes are present in the sample.

Also provided is a system for Raman spectroscopy and fluorescence spectroscopy of a sample of microbeads doped with Raman-active-small-molecules (RASM s), comprising:
a spectrometer;
a light source comprising a first laser and a second laser, the first laser configured to excite Raman scattering from the sample with signature peaks at a first wavelength and the second laser configured to generate an emission spectrum from the sample with wavelengths higher than the first wavelength;
a flowcell through which the sample is passed during spectroscopy of the sample;
telescopic lenses configured to collimate and expand laser beams emitted by the first and second lasers;
reflective lenses for directing the laser beams to the flowcell;
an objective lens configured to focus an emitted Raman signal and an emitted fluorescence frequency spectrum from the sample resulting from illumination of the sample in the flowcell by the laser beams;
a dichroic beam splitter configured to direct the emitted Raman signal and the emitted fluorescence frequency spectrum to the objective lens;
a pin hole through which the focused Raman signal and the fluorescence frequency spectrum are passed; and
relay lenses positioned after the pinhole for directing the Raman signal and the fluorescence frequency spectrum to the spectrometer, the spectrometer comprising a diffraction grating that filters the Raman signal from the fluorescence frequency spectrum so that both the Raman signal and the fluorescence frequency spectrum are detected by the spectrometer.

### BRIEF DESCRIPTION OF THE DRAWINGS

FIGS. 1A-1C: 1A) A large number of Raman-active-small-molecules (RASMs) can be efficiently concentrated in polymer microbeads due to non-covalent interactions; 1B) Using two dimensions of optical barcoding-brightness barcoding and spectral barcoding-manifold unique beads can be generated; 1C) Assuming: i) each one of the RASM can be doped into microbeads at M different concentrations, resulting in microbeads that have M brightness levels (brightness barcoding); ii) a combination of n RASMs can be doped together into one microbead and the Raman signal of each RASM in the same microbead can be detected individually (spectral barcoding), then the total possible number of unique “Vibrant MicroBeads” (a laboratory name for the “barcoded” RASM-loaded microbeads disclosed herein) would be M n−1. If 5 brightness barcodings and 10 spectral barcodings were considered, the resulting unique Vibrant MicroBeads would be 5¹⁰⁻¹=9,765,624 flavors.

FIG. 2: shows massive multiplexity is the direct result of the fact that RASMs can have individually resolvable Raman spectra. In comparison, most fluorescence-based techniques only have about three spectrally resolvable fluorescence dyes usable at the same time, greatly limiting the multiplexity. The red alkyne bonds represent the presence of ¹³C.

FIGS. 3A-3B: 3A shows an annotated image of a mixture of 96 flavors of Vibrant MicroBeads by Stimulated Raman Scattering microscopy. The image was composed of a stack of images each taken at one of the featuring Raman peaks. 3B indicates a spectral analysis of each Vibrant MicroBead and the density plot was generated by plotting the intensity at each Raman shift in a pairwise manner.

FIGS. 4A-4C: Exemplary detection of various mock virus nucleic acid sequence detection including A) optional CRSIPR technique to increase specificity; B) indication of optical barcoding for different mock viral sequences (left panel, barcoded RASM microbeads) and positive detection of sequences (right panel; fluorophore); C) predicted detection fidelity-see FIG. 5 for results.

FIG. 5: Dozens of synthetic targets were synthesized as mock viral genomes across six viral families. Out of 17 mock viral genomes tested, an accuracy of ˜95% was achieved.

FIGS. 6A-6B: 6A: Massively multiplexed fluorescence-linked immunosorbent assay for peptide and protein detection. In a non-limiting example, a certain antibody or aptamer is bioconjugated to the surface of Vibrant MicroBeads. Then different Vibrant MicroBeads with different antibodies can be mixed for the multiplex detection of peptides or protein. 6B: Preliminary tests had been carried out and the sensitive detection limit was realized as low as 5 pg/ml.

FIG. 7: Raman spectrum from a single 5 μm Vibrant MicroBead. Even when the exposure time was as short as 3 ms, the signal-to-noise ratio of the RASMs peak is as high as about 50, making it possible for high throughput detection.

FIGS. 8A-8E: 8A) Exemplary spontaneous Raman flow cytometry setup design. 8B) Shows the schematics of the concept. 8C) Shows a combined Raman and fluorescence spectra of a Vibrant MicroBead with a fluorescence signal attached to it. 8D) For the liquid flow cell system, a microfluidic device featuring a single straight channel (300 μm rectangular cross section) and acoustic focusing powered by a piezo chip vibrating at 2.49 Mhz was used. 8E) Detection with a microwell array. The microwell array is designed so that each microwell can only hold one microbead at a time. Therefore, by scanning the microwell array where the microbeads have settled down, the signal from each individual bead can be acquired efficiently and maintenance-free.

FIGS. 9A-9B: 9A & 9B together show a data matrix summarizing the validation results where 124 viral species and 9 control samples (list in description hereinbelow) were subject to the tests described herein (tested species on left, and detected species on bottom).

FIG. 10: Shows a data matrix summarizing the validation results for an upper respiratory infection panel, tested with inactivated real viruses and bacteria (tested species on left, and detected species on bottom).

FIG. 11: Shows the detection sensitivity of the assays described herein. Strong signal was recorded even the sample concentration was down to 2 aM. The negligible signal from the control group also indicates high specificity.

FIG. 12: A fluorescence dye, Alexa647, was diluted to 100 nM in DMSO and subject to signal acquisition described in FIG. 8. Raman peaks from the solvent are clearly visible, with no elevated background from fluorescence interference. Above 4000 cm-1 (˜675 nm with 532 nm excitation laser wavelength), fluorescence emission from Alexa647 can recorded with very high signal-to-noise ratio.

### DETAILED DESCRIPTION

A method is provided for detecting the presence of one or more different analytes present in a sample comprising:
(a) contacting the sample with a plurality of bead types, each bead type comprising:
one, or more, types of Raman-active small molecule(s), each at a predefined concentration in the bead, and
a binding molecule, affixed thereon, specific for one of each of said one or more different analytes;
wherein each bead type differs from the remaining bead types of the plurality in (i) concentration of Raman-active small molecule(s), (ii) spectrum or spectra of Raman-active small molecule(s), or (ii) both concentration and spectrum/spectra pattern of Raman-active small molecule(s);
(b) removing unbound analytes or unbound beads; and
(c) determining, with Stimulated Raman Scattering (SRS) or Spontaneous Raman Scattering, which bead type(s) having an analyte bound thereto are present after step (b) so as to thereby determine which type(s) of analyte are bound,
thereby detecting which of one or more different analytes are present in the sample.

In embodiments, each bead type comprises a polystyrene particle. In embodiments, the beads are paramagnetic microbeads.

In embodiments, oil-in-water emulsion droplets are used, in place of beads, where a number of different active reactions in the droplet are to be monitored in parallel, and the barcoding serves as the identifier of the identity of the reactions. In embodiments, any substance of substrate that the RASMs can partition stably into can be used in place of beads in the methods described herein, mutatis mutandis.

In embodiments, all bead types are of the same size, or are all of about the same size. In embodiments, each bead type of the plurality has an average diameter of at least 1.0 μm. In embodiments, each bead type of the plurality has an average diameter of 3.0 μm. In embodiments, each bead type of the plurality has an average diameter of 5.0 μm. In embodiments, each bead type of the plurality has an average diameter of 10.0 μm. In embodiments, each bead type of the plurality has an average diameter of not less than 1.0 μm. In embodiments, each bead type of the plurality has an average diameter of not less than 750 nm. In regard to about the same size, the term about means up to 10% more or up to 10% less in diameter to the reference diameter.

In embodiments, the types of bead are further barcoded by size difference. In embodiments, size barcoding is read by taking images and measuring the diameter of each microbead. In embodiments, size barcoding is read quantitatively by measuring the forward and backward scattering (since the scattering strength is dependent on their sizes).

In embodiments, each bead type of the plurality has peak Raman shift at a predetermined stimulation wavelength at least 10 cm⁻¹ less, or 10 cm⁻¹ more, than the peak Raman shift of all the other bead types in the plurality.

Raman-active small molecules are a result of a molecular vibration causing a change in polarizability of the molecule. The symmetrical stretch out and then in can be detected by Raman spectroscopy.

In embodiments, the Raman-active small molecules are alkyne-containing. In embodiments, the Raman-active small molecules are organic and do not exceed a molecular weight of 350 g/mol. In embodiments, the Raman-active small molecules are organic and do not exceed a molecular weight of 310 g/mol. In embodiments, the RASMs are based on one or more Carbow dyes.

In embodiments, the Raman-active small molecules comprise one or more of the following, wherein “*” adjacent to an alkyne carbon atom indicates presence of a 13C isotope:

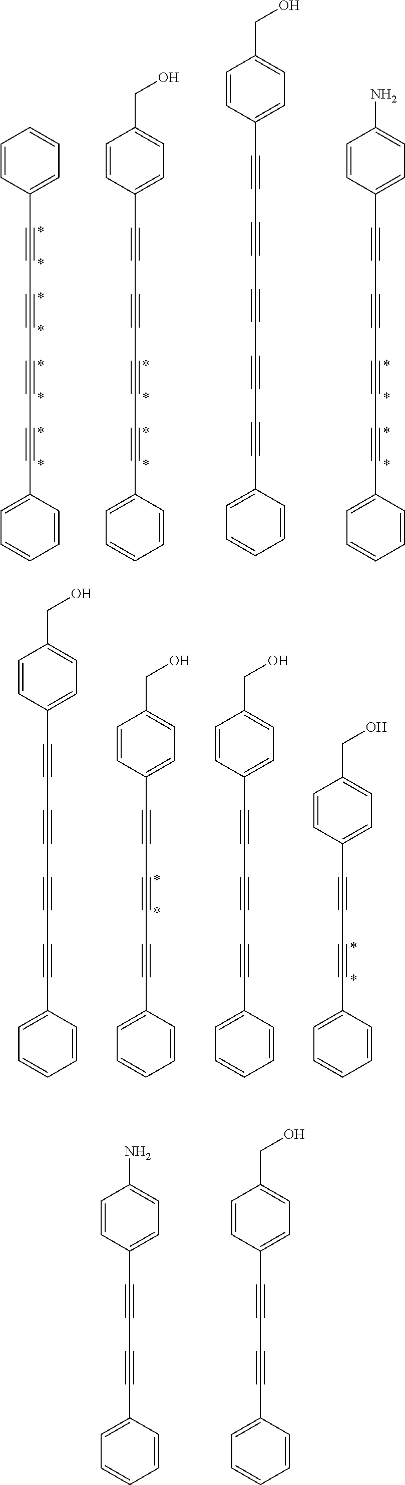

In embodiments, the Raman-active small molecules comprise one or more of the following, wherein “*” adjacent to an alkyne carbon atom indicates presence of a 13C isotope

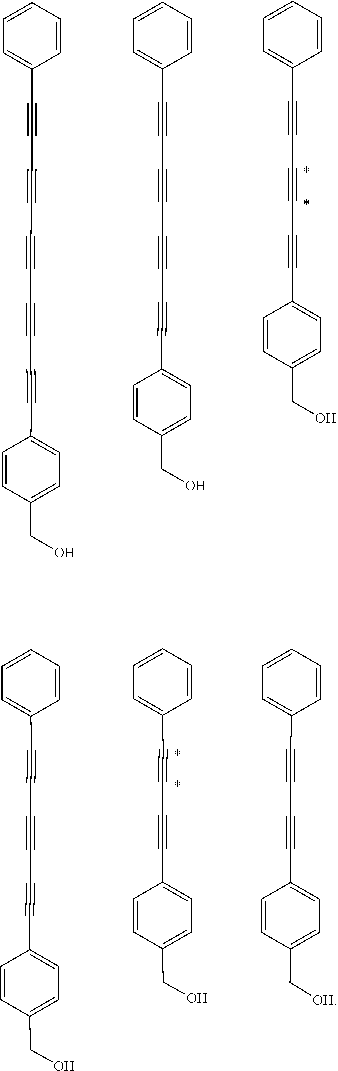 [SMILES: OCc1ccc(C#CC#CC#CC#CC#Cc2ccccc2)cc1 ; OCc1ccc(C#CC#CC#CC#Cc2ccccc2)cc1 ; OCc1ccc(C#CC#CC#Cc2ccccc2)cc1 ; OCc1ccc(C#CC#CC#Cc2ccccc2)cc1 ; OCc1ccc(C#CC#Cc2ccccc2)cc1 ; OCc1ccc(C#CC#Cc2ccccc2)cc1]

In embodiments, the plurality of beads comprises 5 or more different brightness levels. In embodiments, the plurality of beads comprises 10 or more different brightness levels. In embodiments, the plurality of beads comprises 15 or more different brightness levels.

In embodiments, the plurality of beads comprises at least 90 different bead types. In embodiments, the plurality of beads comprises at least 100 different bead types. In embodiments, the plurality of beads comprises at least 500 different bead types. In embodiments, the plurality of beads comprises at least 1000 different bead types. In embodiments, the plurality of beads comprises at least 10,000 different bead types. In embodiments, the plurality of beads comprises at least 100,000 different bead types.

In embodiments, wherein the one or more different analytes are nucleic acids.

In embodiments, the one or more different analytes are peptides.

In embodiments, the one or more different analytes are proteins.

In embodiments, the one or more different analytes are antibodies.

In embodiments, the one or more different analytes are lipids or carbohydrates.

In embodiments, the binding molecule specific for one of each of said one or more different analytes comprises a nucleic acid. In embodiments, the binding molecule specific for one of each of said one or more different analytes comprises an aptamer. In embodiments, bead types are bound to a surface or a sample holder. In embodiments, a bead type is bound by being affixed to a solid surface. In embodiments, a bead type is bound by being restricted in movement within a spatial location, e.g., within an individual well of a sample holder which has an opening aperture smaller than the bead type diameter.

In embodiments, the binding molecule specific for one of each of said one or more different analytes comprises a peptide or antibody or antibody fragment.

In embodiments, the one or more different analytes comprise a nucleic acid sequence found in a pathogen.

In embodiments, the pathogen is a pathogen of a mammal.

In embodiments, the pathogen is a virus or a bacteria.

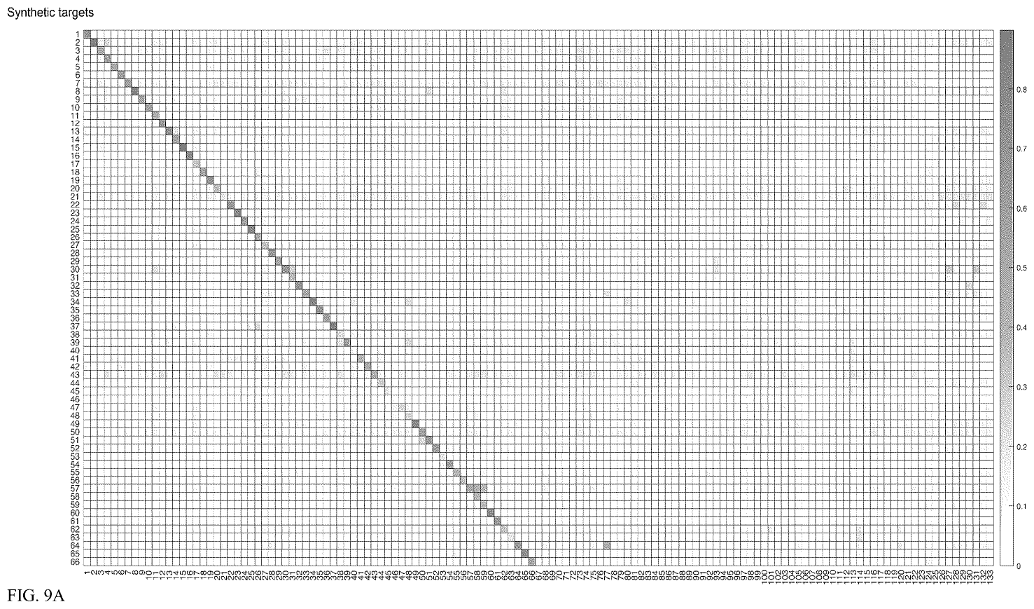

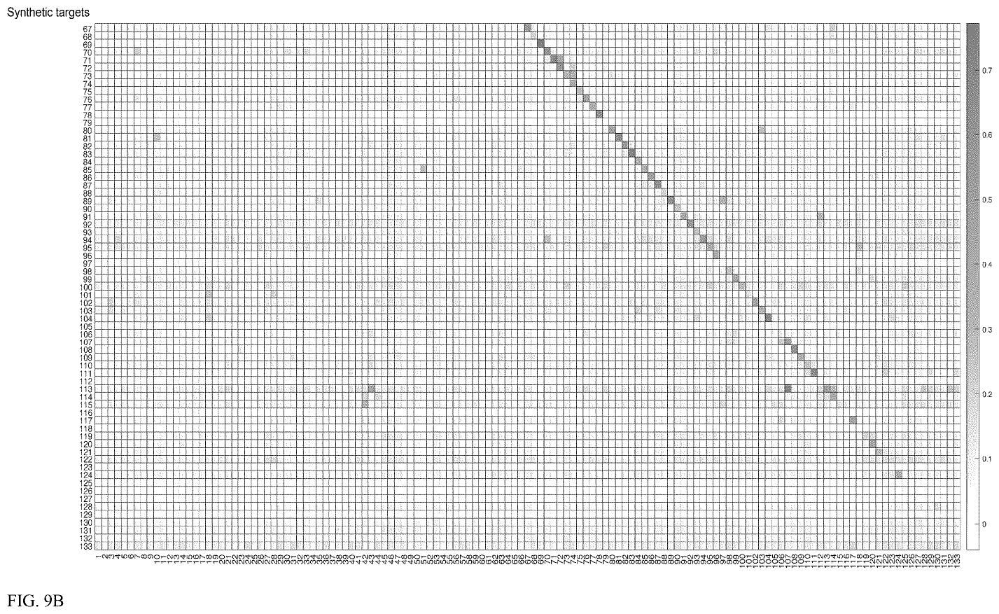

In embodiments, the analytes are nucleic acids from one or more, or a subset of, the pathogens listed in FIG. 9.

In embodiments, the analytes comprise nucleic acids from human respiratory viral pathogens. For example: Adenovirus, Coronavirus HKU1, Coronavirus NL63, Coronavirus 229E, Coronavirus OC43, Human Metapneumovirus, Human Rhinovirus/Enterovirus, Influenza A, Influenza A/H1, Influenza A/H3, Influenza A/H1-2009, Influenza B, Parainfluenza Virus 1, Parainfluenza Virus 2, Parainfluenza Virus 3, Parainfluenza Virus 4, Respiratory Syncytial Virus.

In embodiments, the predefined nucleic acids are from human respiratory bacterial pathogens. For example, Bordetella pertussis, Chlamydophila pneumoniae, Mycoplasma pneumoniae.

In embodiments, the analytes comprise nucleic acids from human gastrointestinal viral pathogens. For example: Adenovirus F40/41, Astrovirus, Norovirus GI/GII, Rotavirus A, Sapovirus (I, II, IV, and V).

In embodiments, the analytes comprise nucleic acids from human gastrointestinal bacterial pathogens. For example: Campylobacter (jejuni, coli and upsaliensis), Clostridium difficile (Toxin A/B), Plesiomonas shigelloides, Salmonella, Yersinia enterocolitica, Vibrio (parahaemolyticus, vulnificus and cholerae), Vibrio cholerae, Diarrheagenic E. coli/Shigella, Enteroaggregative E. coli (EAEC), Enteropathogenic E. coli (EPEC), Enterotoxigenic E. coli (ETEC) It/st, Shiga-like toxin-producing E. coli (STEC) stx1/stx2, E. coli 0157, Shigella/Enteroinvasive E. coli (EIEC).

In embodiments, the analytes comprise nucleic acids from human gastrointestinal parasitic pathogens. For example: Cryptosporidium, Cyclospora cayetanensis, Entamoeba histolytica, Giardia lamblia.

In embodiments, the analytes comprise nucleic acids from human blood pathogens. For example: gram negative bacteria or gram positive bacteria which are pathogenic. For example: Enterococcus, Listeria monocytogenes, Staphylococcus, Streptococcus, Staphylococcus aureus, Streptococcus agalactiae, Streptococcus pneumoniae, Streptococcus pyogenes. For example: Acinetobacter baumannii, Haemophilus influenzae, Neisseria meningitidis, Pseudomonas aeruginosa, Enterobacteriaceae, Enterobacter cloacae complex, Escherichia coli, Klebsiella oxytoca, Klebsiella pneumoniae, Proteus, Serratia marcescens. In embodiments, the predefined nucleic acids are from human blood pathogens which are yeast. For example: Candida albicans, Candida glabrata, Candida krusei, Candida parapsilosis, Candida tropicalis.

In embodiments, the analytes comprise nucleic acids from CNS pathogens, e.g., as found in CSF. For example, bacterial CNS pathogens such as: Escherichia coli K1, Haemophilus influenzae, Listeria monocytogenes, Neisseria meningitidis, Streptococcus agalactiae, Streptococcus pneumoniae. For example, viral CNS pathogens such as: Cytomegalovirus (CMV), Enterovirus, Epstein-Barr virus (EBV), Herpes simplex virus 1 (HSV-1), Herpes simplex virus 2 (HSV-2), Human herpesvirus 6 (HHV-6), Human parechovirus, Varicella zoster virus (VZV). For example, yeast CNS pathogens such as: Cryptococcus gattii, Cryptococcus neoformans.

The analyzed sample can be any analyte of interest from a sample including, for example, DNA, RNA, various mixtures of DNA and RNA. The sample can be, or be derived from, biofluids, blood, serum, urine, dried blood, cell growth media, lysed cells, beverages or food, and can include environmental samples such as water, air or soil. Samples include, without limitation, saliva, blood, CSF, mucus, nasal discharge, GI samples/stool samples, plasma, urine, sweat, and swabbed fluids from, e.g., mouth, lung, vagina, colon.

In embodiments, the analytes comprise nucleic acids from pathogens. In embodiments, the pathogen(s) are human pathogens.

Embodiments of human bacterial pathogens:
Actinomyces israelii
Bacillus anthracis
Bacteroides fragili
Bordetella pertussis
Borrelia
B. burgdorferi
B. garinii
B. afzelii
B. recurrentis
Brucella
B. abortus
B. canis
B. melitensis
B. suis
Campylobacter jejuni
Chlamydia
C. pneumoniae
C. trachomatis
Chlamydophila psittaci
Clostridium
C. botulinum
C. difficile
C. perfringens
C. tetani
Corynebacterium diphtheriae
Ehrlichia
E. canis
E. chaffeensis
Enterococcus
E. faecalis
E. faecium
Escherichia
E. coli (generally)
Enterotoxigenic E. coli (ETEC)
Enteropathogenic E. coli
Enteroinvasive E. coli (EIEC)
Enterohemorrhagic (EHEC), including E. coli 0157: H7
Francisella tularensis
Haemophilus influenzae
Helicobacter pylori
Klebsiella pneumoniae
Legionella pneumophila
Leptospira species
Listeria monocytogenes
Mycobacterium
M. leprae
M. tuberculosis
Mycoplasma pneumoniae
Neisseria
N. gonorrhoeae
N. meningitidis
Pseudomonas aeruginosa
Nocardia asteroides
Rickettsia rickettsii
Salmonella
S typhi
S. typhimurium
Shigella
S. sonnei
S. dysenteriae
Staphylococcus aureus
epidermidis
saprophyticus
Streptococcus agalactiae
pneumoniae
pyogenes
viridans
Treponema pallidum subspecies pallidum
Vibrio cholerae
Yersinia pestis.

Embodiments of human viral pathogens:
A deno-associated virus
Aichi virus
Australian bat lyssavirus
Banna virus
Barmah forest virus
BK polyomavirus
Bunyamwera virus
Bunyavirus La Crosse
Bunyavirus snowshoe hare
Cercopithecine herpesvirus
Chandipura virus
Chikungunya virus
Cosavirus A
Cowpox virus
Coxsackievirus
Crimean-Congo hemorrhagic fever virus
Dengue virus
Dhori virus
Dugbe virus
Duvenhage virus
Eastern chimpanzee simian foamy virus
Eastern equine encephalitis virus
Ebolavirus
Echovirus
Encephalomyocarditis virus
Epstein-Barr virus
European bat lyssavirus
GB virus C/Hepatitis G virus
Hantaan virus
Hendra virus
Hepatitis A virus
Hepatitis B virus
Hepatitis C virus
Hepatitis delta virus
Hepatitis E virus
Horsepox virus
Human adenovirus
Human astrovirus
Human coronavirus
Human cytomegalovirus
Human enterovirus
Human herpesvirus 1
Human herpesvirus 2
Human herpesvirus 6
Human herpesvirus 7
Human herpesvirus 8
Human immunodeficiency virus
Human papillomavirus 1
Human papillomavirus
Human papillomavirus 2
Human parainfluenza
Human parvovirus B 19
Human respiratory syncytial virus
Human rhinovirus
Human SARS coronavirus
Human T-lymphotropic virus
Human torovirus
Influenza A virus
Influenza B virus
Influenza C virus
Isfahan virus
Japanese encephalitis virus
JC polyomavirus
Junin arenavirus
KI Polyomavirus
Lagos bat virus
Lake Victoria marburgvirus
Langat virus
Lassa virus
Louping ill virus
Lymphocytic choriomeningitis virus
Machupo virus
Mammalian orthorubulavirus 5 (Simian virus 5)
Mayaro virus
Measles virus
Merkel cell polyomavirus
MERS coronavirus
Mokola virus
Molluscum contagiosum virus
Monkeypox virus
Mumps virus
Murray valley encephalitis virus
New York virus
Nipah virus
Norwalk virus
O'nyong-nyong virus
Orf virus
Oropouche virus
Pichinde virus
Poliovirus
Punta toro phlebovirus
Puumala virus
Rabies virus
Rift valley fever virus
Rosavirus A
Ross river virus
Rotavirus A
Rotavirus B
Rotavirus C
Rubella virus
Sagiyama virus
Salivirus A
Sandfly fever Naples phlebovirus (Toscana virus)
Sandfly fever sicilian virus
Sapporo virus
SARS coronavirus 2
Semliki forest virus
Seoul virus
Simian foamy virus
Sindbis virus
Southampton virus
St. louis encephalitis virus
Tick-borne powassan virus
Torque teno virus
Uukuniemi virus
Vaccinia virus
Varicella-zoster virus
Variola virus
Venezuelan equine encephalitis virus
Vesicular stomatitis virus
West Nile virus
Western equine encephalitis virus
WU polyomavirus
Y aba monkey tumor virus
Y aba-like disease virus
Yellow fever virus
Zika virus.

In embodiments, the binding molecules specific for one of each of said one or more different analytes comprises a nucleic acid and the different analytes comprise single-nucleotide polymorphisms (SNP) of one or more loci.

In embodiments, the one or more different analytes are nucleic acids and the binding molecule specific for one of each of said one or more nucleic acids to be detected comprises a complementary nucleic acid capable of hybridizing with the analyte nucleic acid.

In embodiments, the method further comprises performing one or more cycles of polymerase chain reaction (PCR) on the analyte nucleic acids of the sample prior to step (a) with one or more primer sequence pairs comprising a forward primer and a reverse primer, the sequence of each of which primers is adjacent to, or flanks, a sequence of the target nucleic acid to which the hybridizing nucleic acid hybridizes.

In embodiments, the method further comprises denaturing double-stranded amplicons resulting from the PCR into single-stranded nucleic acids.

In embodiments, the forward and reverse primers are ended with a repeating 5′ phosphorothioate and the method further comprises contacting the PCR products with a CRISPR cas9 nuclease so as to cleave off 5′ phosphorothioate, thus permitting digestion by lambda-exonuclease so as to form single-stranded DNA.

In embodiments, the reverse primer, or forward primer, of each pair comprises additionally a nucleic acid sequence capable of binding a nucleic acid detection probe that comprises a fluorophore.

In embodiments, the method further comprises contacting the single-stranded nucleic acids with the plurality of bead types and a nucleic acid detection probe comprising a fluorophore, under conditions permitting hybridization of single-stranded nucleic acids to complementary nucleic acid binding molecules affixed on the bead, and hybridization of the nucleic acid detection probes to the single-stranded nucleic acids, and then washing to remove nucleic acids not bound to a bead, wherein the presence of the fluorophore co-located with a type of bead indicates the presence of the nucleic acid analyte in the sample and wherein the type of that bead determined in step (c) indicates the specific analyte present.

In embodiments, the nucleic acid analytes are nucleic acids specific to a viral pathogen or bacterial pathogen.

In embodiments, the one or more different analytes are proteins, wherein the binding molecule specific for one of each of said one or more proteins to be detected comprises a peptide, aptamer, antibody or antibody fragment capable of hybridizing with the analyte protein.

In embodiments, prior to step (c) the bound analyte is contacted with a second peptide, second aptamer, second antibody or second antibody fragment, labelled with a biotin or streptavidin, capable of hybridizing with the analyte protein, under conditions permitting the said second peptide, second aptamer, second antibody or second antibody fragment to bind the analyte, and further comprising contacting the biotin or streptavidin with a fluorophore-labelled streptavidin, or fluorophore-labelled biotin, respectively, and then washing to remove unbound reagents.

In embodiments, the presence of the fluorophore co-located with a type of bead indicates the presence of a protein analyte in the sample and wherein the type of that bead determined in step (c) indicates the specific protein analyte present.

In embodiments, the SRS is performed using a 532 nm laser.

In embodiments, the fluorophore is a far-red fluorophore. In embodiments, the fluorophore is a Cy5. In embodiments, the fluorophore, with an excitation peak at 651 nm and an emission peak at 670 nm.

In embodiments, the fluorophore has an emission maxima greater than 660 nm.

In embodiments, the fluorophore is excited with a 660 nm laser.

In embodiments, presence of the fluorophore is detected by an imaging device.

In embodiments where the analytes are nucleic acids, analyte levels as low as 20 aM, 5 aM or 2 aM can be detected. In embodiments where the analytes are peptides or proteins, analyte levels as low as 20 pg/ml, 10 pg/ml or 5 pg/ml can be detected.

Also provided is a system for Raman spectroscopy and fluorescence spectroscopy of a sample of microbeads doped with Raman-active-small-molecules (RASM s), comprising:
a spectrometer;
a light source comprising a first laser and a second laser, the first laser configured to excite Raman scattering from the sample with signature peaks at a first wavelength and the second laser configured to generate an emission spectrum from the sample with wavelengths higher than the first wavelength;
a sample holder through which the sample is passed during spectroscopy of the sample;
telescopic lenses configured to collimate and expand laser beams emitted by the first and second lasers;
reflective lenses for directing the laser beams to the sample holder;
an objective lens configured to focus an emitted Raman signal and an emitted fluorescence frequency spectrum from the sample resulting from illumination of the sample in the sample holder by the laser beams;
a dichroic beam splitter configured to direct the emitted Raman signal and the emitted fluorescence frequency spectrum to the objective lens;
a pin hole through which the focused Raman signal and the fluorescence frequency spectrum are passed; and
relay lenses positioned after the pinhole for directing the Raman signal and the fluorescence frequency spectrum to the spectrometer, the spectrometer comprising a diffraction grating that filters the Raman signal from the fluorescence frequency spectrum so that both the Raman signal and the fluorescence frequency spectrum are detected by the spectrometer.

In embodiments, the sample holder comprises a microfluidic device. In embodiments, the sample holder comprises a flowcell, a microarray or a glass substrate. In embodiments, the glass substrate is a glass slide, such as a glass microscope slide.

In embodiments, the system further comprises an inverted microscope that comprises the dichroic beam splitter and the objective lens.

In embodiments, the system further comprises relay lenses between the pinhole and the spectrometer.

In embodiments, the system further comprises at least one of a long-pass filter and a notch filter between the relay lenses.

In embodiments, the system further comprises a photodiode configured to detect forward scattering of the laser beams to trigger the spectrometer.

In embodiments, the methods described herein are performed using a system described herein.

In embodiments, the plurality of bead types is affixed to a surface. In embodiments, the analytes to be analyzed are affixed to a surface

A method is provided for detecting the presence of one or more different analytes present in a sample comprising:
(a) contacting the sample with a plurality of types of discrete portions of a substrate, wherein Raman-active small molecule(s) can stably partition into said discrete portions of a substrate, each discrete portion of a substrate comprising:
one, or more, types of Raman-active small molecule(s), each at a predefined concentration in each type of discrete portion, and
a binding molecule, affixed thereon or present therein, specific for one of each of said one or more different analytes;
wherein each type of discrete portion differs from the remaining types of discrete portions of the plurality in (i) concentration of Raman-active small molecule(s), (ii) spectrum or spectra of Raman-active small molecule(s), or (ii) both concentration and spectrum/spectra pattern of Raman-active small molecule(s);
(b) removing unbound analytes; and
(c) determining, with Stimulated Raman Scattering (SRS) or Spontaneous Raman Scattering, which type(s) of discrete portion having an analyte bound thereto are present after step (b) so as to thereby determine which type(s) of analyte are bound,
thereby detecting which of one or more different analytes are present in the sample.

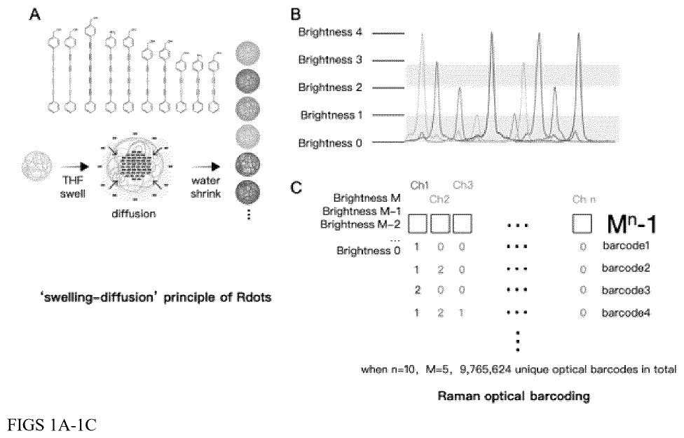

As has been demonstrated in earlier work (See world wide web at doi.org/10.1038/s41467-021-21570-0) a large number of Raman-active-small-molecules (RASM s) can be efficiently doped into polymer nanoparticles due to non-covalent interactions (FIG. 1A). The Raman scattering signal from doped particles is therefore increased as a function of the intra-particle concentration of those RASMs. The signal enhancement factor is proportional to the volume of the particles as larger particles can contain a greater number of RASMs. Also, the Raman spectra of RASMs remain unchanged when doped into particles, making them ideal for scaling up as one only needs to develop RASMs to expand the multiplexity, which is a much easier task than, for example, developing new polymer chemistry.

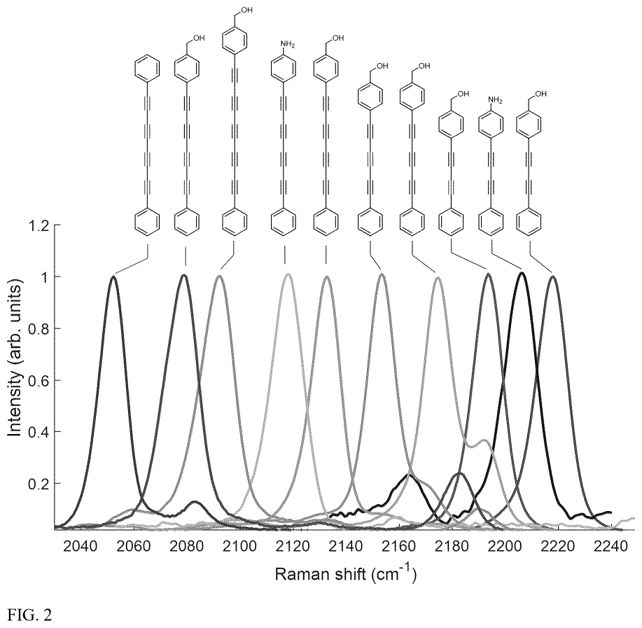

Herein, to greatly expand the multiplexity from the currently available dozens of RASMs, a combinatorics strategy was adopted to generate millions of flavors of “Vibrant MicroBeads” (a laboratory name for the disclosed barcoded RASM beads) each having a unique Raman optical barcode (FIGS. 1B-1C). This strategy was achieved by using two dimensions of optical barcoding: brightness barcoding and spectral barcoding. Assuming: 1) each one of the RASM can be doped into microbeads at M different concentrations, resulting in microbeads that have M brightness levels (brightness barcoding); and 2) a combination of n RASMs can be doped together into one microbead and the Raman signal of each RASM in the same microbead can be detected individually (spectral barcoding), then the total possible number of unique Vibrant MicroBeads would be Mⁿ⁻¹. Thus, if 5 brightness barcoding and 10 spectral barcoding were considered, the resulting unique Vibrant MicroBeads would be 5¹⁰⁻¹=9,765,624 flavors (FIG. 1B). This massive multiplexity is the direct result of the fact that these 10 RASMs have individually resolvable Raman spectra (FIG. 2), whereas, for most fluorescence-based techniques, only about 3 spectrally resolvable fluorescence dyes can be used at the same time, greatly limiting the multiplexity.

In addition to the brightness barcoding and spectral barcoding mechanism, size barcoding can also be used in contexts where various sizes of microbeads can be used together to further increase the multiplexity. These size barcoding can either be read out by taking images and directly measuring the diameter of each microbead or by quantitatively measuring the forward and backward scattering as the scattering strength is dependent on their sizes. When size barcoding is made use of, the total multiplexity would be S*(M n−1) where S indicates the number of different sizes.

Since the Raman signal enhancement factor is proportional to the volume of the particles, relatively large micron-sized polystyrene beads (for example, 3 μm; 5 μm, and 10 μm diameter) were chosen as the particle-matrix for maximizing the Raman signal intensity and increasing the throughput. To generate Vibrant MicroBeads, one or combinations of spectrally distinguishable few RASMs of various concentrations were doped into polystyrene beads through a swell-and-diffusion mechanism. For example, in a 5 ml tube, 320 μl 4% w/v polystyrene microbeads and 320 μl RO water were mixed. In another 1.5 ml tube, 40 μl 20× Carbow dye(s) DMSO stock solution and 120 μl THF (Sigma, 401757) were mixed. Then the THF/dye(s) mixture was added to the diluted microbeads to swell the microbeads and incorporate the dyes, followed by brief vortexing and gentle agitation with a rotary wheel for 30 min at room temperature. Dye concentration level and dye identity/mixture for each set of beads being manufactured was chosen beforehand so as to leverage the brightness barcoding and spectral barcoding concepts, respectively. Next, 3 ml 20 mM phosphate buffer (pH 7.3) was slowly added to shrink and trap the dyes in the microbeads. The mixture was then centrifuged at max speed for 2 min to remove any insoluble materials, and the supernatant was collected to yield Vibrant MicroBeads. Washing 3 times using Amicon 30 kDa MWCO filters (Millipore, UFC9030) with at least 10 ml RO water removes excessive organic solvents.

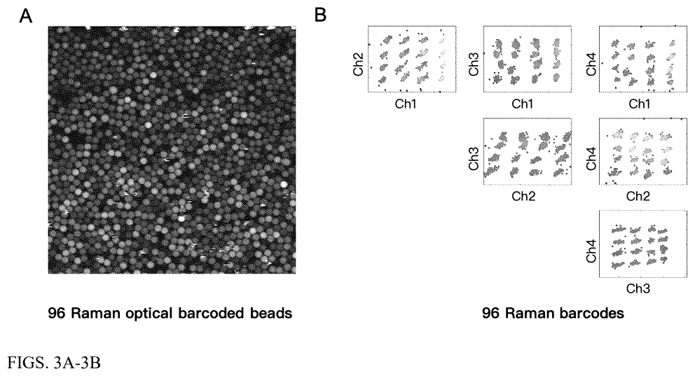

FIG. 3A shows an annotated image of a mixture of 96 flavors of Vibrant MicroBeads by Stimulated Raman Scattering microscopy. The image was composed of a stack of images each taken at one of the featuring Raman peaks. FIG. 3B indicated a spectral analysis of each Vibrant MicroBead and the density plot was generated by plotting the intensity at each Raman shift in a pairwise manner.

Although in the preliminary data herein set only 5 brightness barcoding levels were demonstrated, more levels are possible. The total possible levels are determined by both the intensity distribution of each single Vibrant MicroBead as well as the dynamic range of the detection system.

Besides the polystyrene as the particle-matrix which was demonstrated, it is possible to utilize other materials as long as RASMs can be doped or dissolved in those materials. For example, beads with paramagnetic microbeads can be used as the matrix to generate magnetic Vibrant MicroBeads for easy separation. Oil-water emulsion droplets can also be used if a large number of different active reactions in the droplet are to be monitored in parallel, and the barcoding can serve as the identifier of the identity of the reactions.

The Vibrant MicroBeads were tested in actual use situations, described as follows.

Human virome detection: Infectious diseases are one of the major threats to human health, yet only a small portion of pathogens can be effectively monitored. Among over 1000 viruses that infect vertebrates, only dozens of them can be detected in clinical practice. In addition, most common methods suffer from low throughput (qPCR for instance), or high cost and long turnover (such as sequencing methods). Therefore, a rapid high-throughput method is in demand for viral surveillance, and quick diagnosis. Here such a method capable of diagnosing certain viral infections among all known human virome was designed and tested.

First, a set of DNA hybridization probes, each one of them targeting a viral species, were designed for all viral species with known genomes. Then, each of these hybridization probes was bioconjugated to a predetermined Vibrant MicroBead, respectively. To improve specificity, maximum sequence diversity was ensured among these probes. Also designed were a set of DNA primers that flank these hybridization regions in the viral genome for each viral species. For the reverse primers, a unique sequence overhang was added to the 5′ end (TATCTTAAGTCTTCGCGTG) (SEQ ID NO: 1) to serve as the binding site of the fluorescence detection probe (this could also be attached alternatively to the forward primer). Second, the viral genome was extracted (and reverse transcribed for RNA virus) and then amplified with the primer set by PCR. Third, after denaturing with NaOH, the resulting single-stranded PCR products were mixed with the bioconjugated Vibrant MicroBeads as well as the detection probe, and then acid was added to obtain neutral pH and permit hybridization between the amplified PCR product and the Vibrant MicroBeads. The Vibrant MicroBeads unbound DNA was then washed away. Lastly, the Vibrant MicroBeads were imaged by SRS or detected using a spontaneous Raman flow cytometer. When a virus species was present in the sample, it would be efficiently amplified and hybridized to its corresponding Vibrant MicroBead, and a fluorescence signal could be detected from the surface of the beads, thus detecting both the presence of and the identity of the specific viral nucleic acid in the sample.

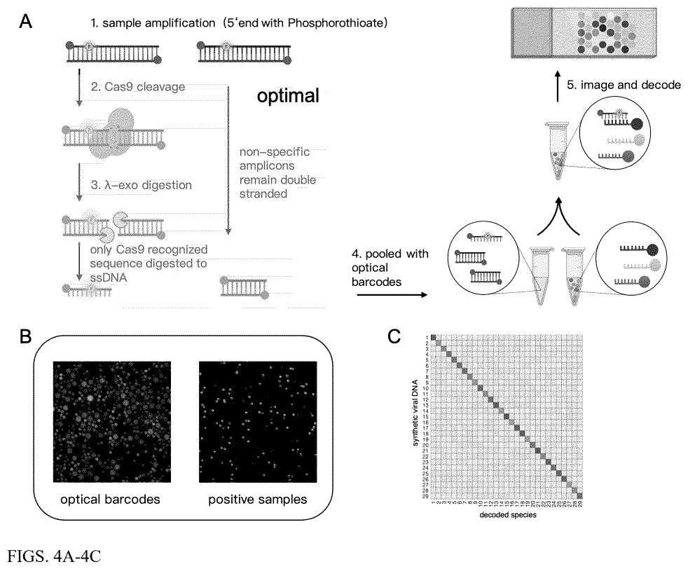

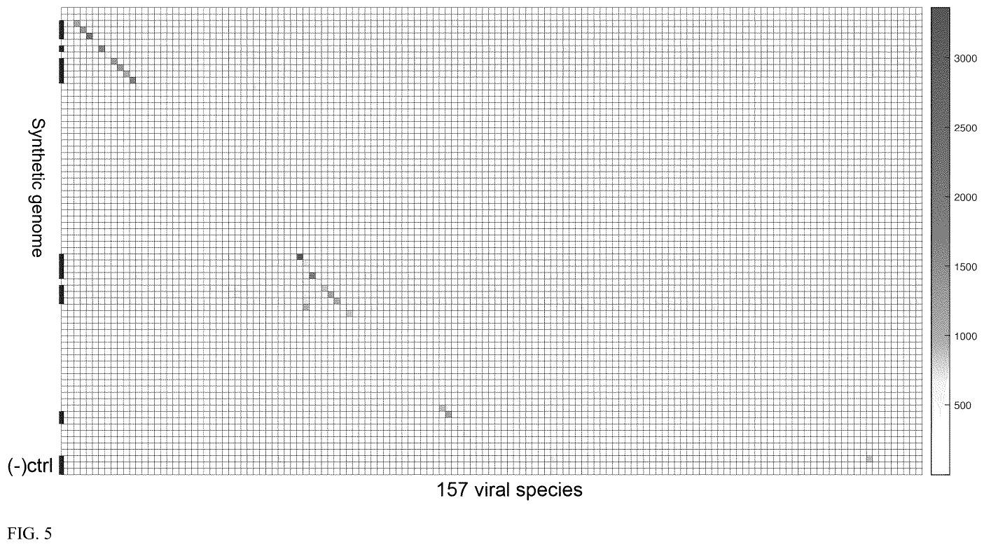

To further increase the specificity, a CRISPR technique combined with exonuclease can be used to generate single-stranded hybridizable DNA in place of the alkaline denaturing. For this, both the forward and reverse primers are ended with repeating 5′ phosphorothioate. Consequently, only the PCR products that are recognized by the CRISPR cas9 nuclease (which cleaves away the 5′ protective phosphorothioate) can be digested by lambda-exonuclease and form single-stranded DNA which can then hybridize to their Vibrant MicroBeads. By doing this method, another checkpoint is realized by the specificity intrinsic to CRISPR to further increase the detection fidelity. See FIGS. 4A-4C.

Synthetic viral genomes were used to demonstrate the performance. In the preliminary test, dozens of synthetic targets were synthesized to mock viral genomes across 6 viral families. Out of 17 mock viral genomes tested, an accuracy of ˜95% accuracy was achieved (FIG. 5).

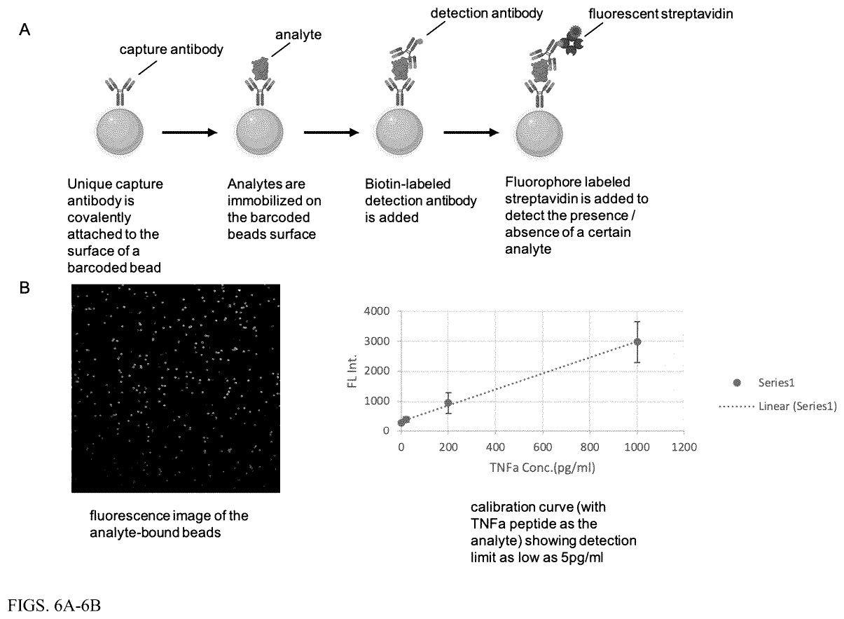

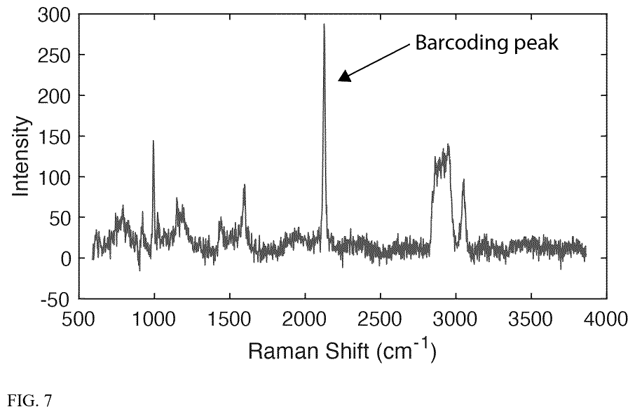

Another application for Vibrant MicroBeads is massively multiplexed fluorescence linked immunosorbent assay for peptide and protein detection. Briefly, a certain antibody or aptamer is bioconjugated to the surface of Vibrant MicroBeads. Then different Vibrant MicroBeads with different antibodies can be mixed for the multiplex detection of peptides or proteins (FIG. 6A-6B). Preliminary tests had been carried out and the sensitive detection limit was realized as low as 5 pg/ml (FIG. 6C).

As an information carrier that contains the identity of the analyte in a more generalized term, Vibrant MicroBeads can be coupled with any kinds of affinity binders such as antibodies and nucleic acids to detect their corresponding ligand in a parallel and multiplexed way. For example, SNP phenotyping is also possible when specific oligo sequences that bind SNP flanking region can be coupled to Vibrant MicroBeads and single nucleotide extension can be introduced to readout the SN P.

Raman flow cytometer: Currently, coherent Raman techniques such as Coherent anti-Stokes Raman spectroscopy (CARS) and Stimulated Raman Scattering spectroscopy (SRS) are widely used in high-speed applications for their orders of magnitude stronger signal compared to spontaneous Raman. However, coherent Raman techniques usually employ ultrafast pulse laser sources which are not readily accessible to most users both economically and technically.

Since the Vibrant MicroBeads possess strong Raman scattering intensity as a result of concentrated RASMs, it is possible to acquire a sufficiently good signal with spontaneous Raman. FIG. 7 shows the Raman spectrum from a single 5 μm Vibrant MicroBead. Even when the exposure time was as short as 3 ms, the signal-to-noise ratio of the RASM s peak is as high as about 50, making it possible for high throughput detection. Assuming the desirable SNR is about 5, then the minimum exposure time can be as short as 0.3 ms, and therefore the throughput can be more than 3000 events per second, which is close to fluorescence-based flow cytometry techniques.

If the same number of events would be acquired with SRS, where laser wavelength needs to be tuned to excite each one of the barcoding Raman peaks, it would usually take 1 min to accumulate 3000 events for each frame of wavelength and thus 7 min in total for 7 peaks for example for the actual acquisition time. Y et the wavelength tuning takes about 2 min every time, so the total time consumed for the same 3000 events would take no less than 21 min. This makes sense since SRS provides high spatial resolution in the sacrifice of throughput, however, in certain scenarios such as barcoding detection discussed here, the spatial information is useless because only the spectral barcoding information of each bead is of interest, the shapes as well as the positions of the beads are not of concern.

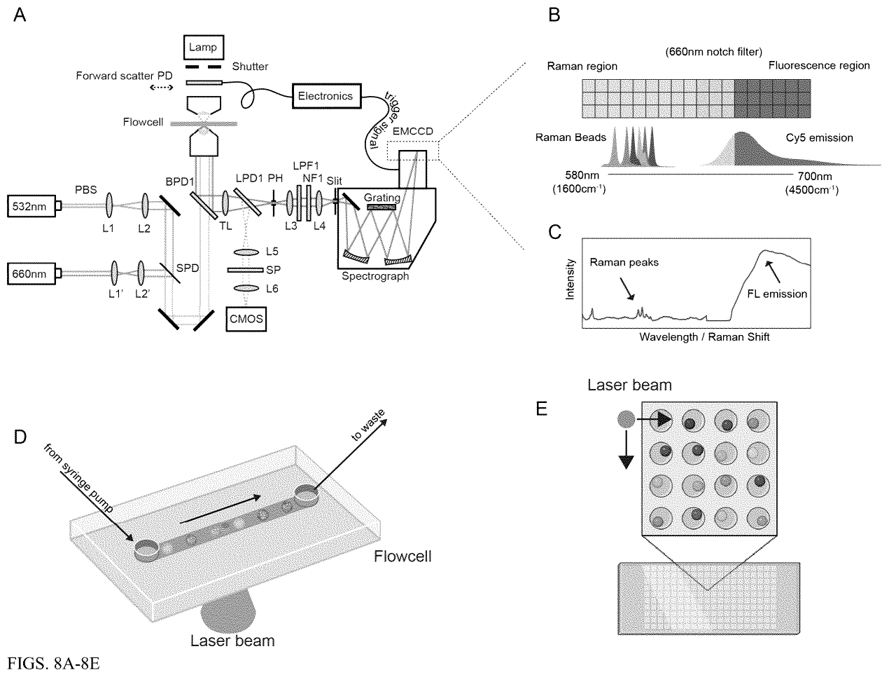

Given that the Raman signal of Vibrant MicroBeads can be detected using spontaneous Raman in high fidelity, a custom spontaneous Raman flow cytometry setup was designed and built (FIG. 8A). In order to read barcoding information and identity information at the same time, the goal of this setup was to acquire Raman scattering signal and fluorescence signal simultaneously on the same detector. This would allow for simpler design as complicated electronics to synchronize Raman and fluorescence signal can be then eliminated. However, this is not common practice because usually the fluorescence signal overwhelms the Raman signal and many techniques such as fluorescence quencher, shifted excitation, FT-Raman, and so on were developed to suppress the fluorescence background so that the Raman signal can be reliably readout.

To solve this problem, a few strategies were adopted herein. Firstly, a 532 laser was used to excite the Raman scattering so that signature peaks from RA SM s (˜2000 cm-1) were around 600 nm. Secondly, a red dye (such as Cy5) was used in fluorescence having emission maximum redder than 660 nm. This way, the emission spectrum is well separated from the Raman peaks (that were around 600 nm) and thus avoiding interfering with them. Thirdly, a diffraction grating with low groove density (600 l/cm) was chosen so that while a good spectral resolution was preserved, both the Raman spectra and fluorescence emission spectra could fit into the same camera frame. It should be noted that this design was especially suitable to be coupled with Vibrant MicroBeads because the Raman peaks were so strong that the relatively weak anti-stokes emission of the fluorescence dye would not affect the detection of Raman scattering signals.

FIG. 8B shows the schematics of the concept. FIG. 8C shows a combined Raman and fluorescence spectra of a Vibrant MicroBead with a fluorescence signal attached to it. Judging from the spectrum, Raman peaks themselves were well resolvable, and they were also well separated from the fluorescence emission signal. The Raman peaks were also background free, as discussed above so the weak anti-stokes emission from the fluorescence dye would not affect Raman signals.

The schematics of an exemplary home-built confocal Raman microscope is shown in FIG. 8A. A 532 nm laser (Samba 532 nm, 400 mW, Cobolt Inc.) and a 660 nm laser (Obis 660 nm 100 mW, Coherent) were used as the light source. The laser beam was first collimated and expanded by the telescope lenses (L1, L2/L1′, L2′, Thorlabs). The expanded beam was then directed to the inverted microscope (IX 71, Olympus) installed with a dichroic beamsplitter (LPD1, LPD02-532RU-25, Semrock). The emitted Raman signal first passed a pinhole (PH, 300 um, Thorlabs) for background suppression and was relayed by two lenses (L3, L4, Thorlabs) before being projected to the spectrometer (Kymera 328i with 600 lines/mm grating blazed at 500 nm, Andor). A long-pass filter (LP, LP03-532RU-25, Semrock) and notch filter (NF658-26, Thorlabs) were installed between the relay lenses to block laser light from Rayleigh scattering. Raman signal was then collected by an EM CCD (Newton970, Andor). For brightfield imaging, a long-pass dichroic (LPD2, FF511-Di01, Semrock) was installed in front of the pinhole and a set of relay lenses (L5, L6) were used to project the brightfield images to the CMOS camera (DCC1645C, Thorlabs). A short-pass filter (SP) was installed to suppress ghost images. A photodiode (PD, SM 05PD3A, Thorlabs) was mounted on a swivel mount installed above the sample stage, and was used to detect forward scattering to trigger the EMCCD acquisition.

Although a microscope base was used in this design, it is not necessary to include the microscope per se. For a simpler design, only the objective would be needed and the microscope base can be replaced by a customized frame that can hold the objective, which is the core of the detection system.

For the liquid flow cell system, a microfluidic device featuring a single straight channel (300 μm rectangular cross section) and acoustic focusing powered by a piezo chip vibrating at 2.49 Mhz was used (FIG. 8D). Other chips or capillary tubes can also be used as long as they provide the capability that can align the particles in the channel. 8E) Detection with a microwell array. The microwell array is designed so that each microwell can only hold one microbead at a time. Therefore, by scanning the microwell array where the microbeads have settled down, the signal from each individual bead can be acquired efficiently and maintenance free.

FIG. 9: shows confirmation validation of the technique. The following viral species (and control samples) are shown, with the numbered rows starting at the top of the left hand side of the display and increasing by number consecutively as one travels down the chart:
1 Adenoviridae_Mastadenovirus_Human_mastadenovirus_B
2 Adenoviridae_Mastadenovirus_Human_mastadenovirus_C
3 Adenoviridae_Mastadenovirus_Human_mastadenovirus_D
4 Adenoviridae_Mastadenovirus_Human_mastadenovirus_E
5 Adenoviridae_Mastadenovirus_Human_mastadenovirus_F
6 Anelloviridae_Betatorquevirus_TTV_like_mini_virus
7 Anelloviridae_Gyrovirus_Avian_gyrovirus_2
8 Anelloviridae_Gyrovirus_Chicken_anemia_virus
9 Anelloviridae_lotatorquevirus_Torque_teno_sus_virus_1a
10 Anelloviridae_lotatorquevirus_Torque_teno_sus_virus_1b
11 Anelloviridae_Torque_teno_Leptonychotes_weddellii_virus_1
12 Anelloviridae_Torque_teno_Leptonychotes_weddellii_virus_2
13 Anelloviridae_Torque_teno_virus
14 Arenaviridae_Arenavirus_M opeia_Lassa_virus_reassortant_29_L
15 Arenaviridae_Mammarenavirus_Argentinian_mammarenavirus_L
16 Arenaviridae_Mammarenavirus_Cali_mammarenavirus_S
17 Arenaviridae_Mammarenavirus_Guanarito_mammarenavirus_L
18 Arenaviridae_Mammarenavirus_Lassa_mammarenavirus_L
19 Arenaviridae_Mammarenavirus_Lymphocytic_choriomeningitis_mammarenavirus_L
20 Arenaviridae_Mammarenavirus_Whitewater_Arroyo_mammarenavirus_S
21 Astroviridae_Mamastrovirus_Mamastrovirus_1
22 Caliciviridae_Sapovirus_Sapporo_virus
23 Coronaviridae_Alphacoronavirus_Human_coronavirus_229E
24 Coronaviridae_Alphacoronavirus_Human_coronavirus_NL63
25 Coronaviridae_Betacoronavirus_Human_coronavirus_HKU1
26 Coronaviridae_Betacoronavirus_Middle_East_respiratory_syndrome_related_coronavirus
27 Coronaviridae_Betacoronavirus_Severe_acute_respiratory_syndrome_related_coronavirus
28 Deltavirus_Hepatitis_delta_virus
29 Filoviridae_Ebolavirus_Sudan_ebolavirus
30 Filoviridae_Ebolavirus_Zaire_ebolavirus
31 Filoviridae_Marburgvirus_Marburg_marburgvirus
32 Flaviviridae_Flavivirus_Bagaza_virus
33 Flaviviridae_Flavivirus_Culex_flavivirus
34 Flaviviridae_Flavivirus_Dengue_virus
35 Flaviviridae_Flavivirus_Japanese_encephalitis_virus
36 Flaviviridae_Flavivirus_Kyasanur_Forest_disease_virus
37 Flaviviridae_Flavivirus_Murray_Valley_encephalitis_virus
38 Flaviviridae_Flavivirus_Saint_Louis_encephalitis_virus
39 Flaviviridae_Flavivirus_Tick_borne_encephalitis_virus
40 Flaviviridae_Flavivirus_Usutu_virus
41 Flaviviridae_Flavivirus_West_Nile_virus
42 Flaviviridae_Flavivirus_Yellow_fever_virus
43 Flaviviridae_Flavivirus_Zika_virus
44 Flaviviridae_Hepacivirus_Hepacivirus_C
45 Flaviviridae_Pegivirus_Pegivirus_A
46 Flaviviridae_Pegivirus_Pegivirus_C
47 Flaviviridae_Pegivirus_Pegivirus_H
48 Hantaviridae_Orthohantavirus_Andes_orthohantavirus_S
49 Hantaviridae_Orthohantavirus_Dobrava_Belgrade_orthohantavirus_L
50 Hantaviridae_Orthohantavirus_Imjin_orthohantavirus_M
51 Hantaviridae_Orthohantavirus_Nova_orthohantavirus_S
52 Hantaviridae_Orthohantavirus_Puumala_orthohantavirus_M
53 Hantaviridae_Orthohantavirus_Seoul_orthohantavirus_L
54 Hantaviridae_Orthohantavirus_Sin_Nombre_orthohantavirus_M
55 Hantaviridae_Orthohantavirus_Thottapalayam_orthohantavirus_S
56 Hantaviridae_Orthohantavirus_Tula_orthohantavirus_S
57 Hepeviridae_Orthohepevirus_Orthohepevirus_A
58 Herpesviridae_Cytomegalovirus_Human_betaherpesvirus_5
59 Herpesviridae_Lymphocryptovirus_Human_gammaherpesvirus_4
60 Herpesviridae_Simplexvirus_Human_alphaherpesvirus_2
61 Herpesviridae_Varicellovirus_Human_alphaherpesvirus_3
62 Nairoviridae_Orthonairovirus_Crimean_Congo_hemorrhagic_fever_orthonairovirus_L
63 Nairoviridae_Orthonairovirus_Nairobi_sheep_disease_orthonairovirus_S
64 Orthomyxoviridae_Alphainfluenzavirus_Influenza_A_virus_1
65 Orthomyxoviridae_Betainfluenzavirus_Influenza_B_virus_1
66 Orthomyxoviridae_Gammainfluenzavirus_Influenza_C_virus_3
67 Papillomaviridae_Alphapapillomavirus_Alphapapillomavirus_4
68 Papillomaviridae_Betapapillomavirus_Betapapillomavirus_1
69 Paramyxoviridae_Avulavirus_Avian_avulavirus_1
70 Paramyxoviridae_Henipavirus_Hendra_henipavirus
71 Paramyxoviridae_Henipavirus_Nipah_henipavirus
72 Paramyxoviridae_Morbillivirus_Canine_morbillivirus
73 Paramyxoviridae_Morbillivirus_Feline_morbillivirus
74 Paramyxoviridae_Morbillivirus_Measles_morbillivirus
75 Paramyxoviridae_Morbillivirus_Rinderpest_morbillivirus
76 Paramyxoviridae_Respirovirus_Bovine_respirovirus_3
77 Paramyxoviridae_Rubulavirus_Human_rubulavirus_2
78 Peribunyaviridae_Orthobunyavirus_Akabane_orthobunyavirus_M
79 Peribunyaviridae_Orthobunyavirus_Bunyamwera_orthobunyavirus_L
80 Peribunyaviridae_Orthobunyavirus_California_encephalitis_orthobunyavirus_L
81 Peribunyaviridae_Orthobunyavirus_Oropouche_orthobunyavirus_L
82 Peribunyaviridae_Orthobunyavirus_Sathuperi_orthobunyavirus_M
83 Peribunyaviridae_Orthobunyavirus_Shuni_orthobunyavirus_M
84 Phenuiviridae_Phlebovirus_Candiru_phlebovirus_L
85 Phenuiviridae_Phlebovirus_Rift_Valley_fever_phlebovirus_L
86 Phenuiviridae_Phlebovirus_SFTS_phlebovirus_L
87 Phenuiviridae_Phlebovirus_Sandfly_fever_Naples_phlebovirus_L
88 Phenuiviridae_Phlebovirus_Uukuniemi_phlebovirus_L
89 Picornaviridae_Aphthovirus_Foot_and_mouth_disease_virus
90 Picornaviridae_Cardiovirus_Cardiovirus_A
91 Picornaviridae_Enterovirus_Enterovirus_A
92 Picornaviridae_Enterovirus_Enterovirus_D
93 Picornaviridae_Enterovirus_Enterovirus_E
94 Picornaviridae_Enterovirus_Rhinovirus_B
95 Picornaviridae_Enterovirus_Rhinovirus_C
96 Picornaviridae_Hepatovirus_Hepatovirus_A
97 Picornaviridae_Kobuvirus_Aichivirus_A
98 Picornaviridae_Parechovirus_Parechovirus_A
99 Pneumoviridae_Metapneumovirus_Avian_metapneumovirus
100 Pneumoviridae_Orthopneumovirus_Human_orthopneumovirus
101 Pneumoviridae_Respiratory_syncytial_virus
102 Polyomaviridae_Alphapolyomavirus_Human_polyomavirus_5
103 Polyomaviridae_Betapolyomavirus_Macaca_mulatta_polyomavirus_1
104 Poxviridae_Orthopoxvirus_Cowpox_virus
105 Poxviridae_Parapoxvirus_Orf_virus
106 Reoviridae_Orthoreovirus_Mammalian_orthoreovirus_L2
107 Reoviridae_Orthoreovirus_Mammalian_orthoreovirus_L3
108 Reoviridae_Orthoreovirus_Mammalian_orthoreovirus_M1
109 Reoviridae_Orthoreovirus_Mammalian_orthoreovirus_M2
110 Reoviridae_Orthoreovirus_Mammalian_orthoreovirus_M3
111 Reoviridae_Orthoreovirus_Mammalian_orthoreovirus_S1
112 Reoviridae_Orthoreovirus_Mammalian_orthoreovirus_S3
113 Reoviridae_Orthoreovirus_Mammalian_orthoreovirus_S4
114 Reoviridae_Rotavirus_Rotavirus_A_10
115 Reoviridae_Rotavirus_Rotavirus_B_1
116 Reoviridae_Rotavirus_Rotavirus_C_3
117 Reoviridae_Seadornavirus_Banna_virus_12
118 Retroviridae_Deltaretrovirus_Primate_T_lymphotropic_virus_1
119 Retroviridae_Lentivirus_Human_immunodeficiency_virus_1
120 Retroviridae_Lentivirus_Simian_immunodeficiency_virus
121 Rhabdoviridae_Lyssavirus_European_bat_1_lyssavirus
122 Rhabdoviridae_Lyssavirus_Rabies_lyssavirus
123 Rhabdoviridae_Vesiculovirus_Indiana_vesiculovirus
124 Rhabdoviridae_Vesiculovirus_New_Jersey_vesiculovirus
125 control
126 control
127 control
128 control
129 control
130 control
131 control
132 control

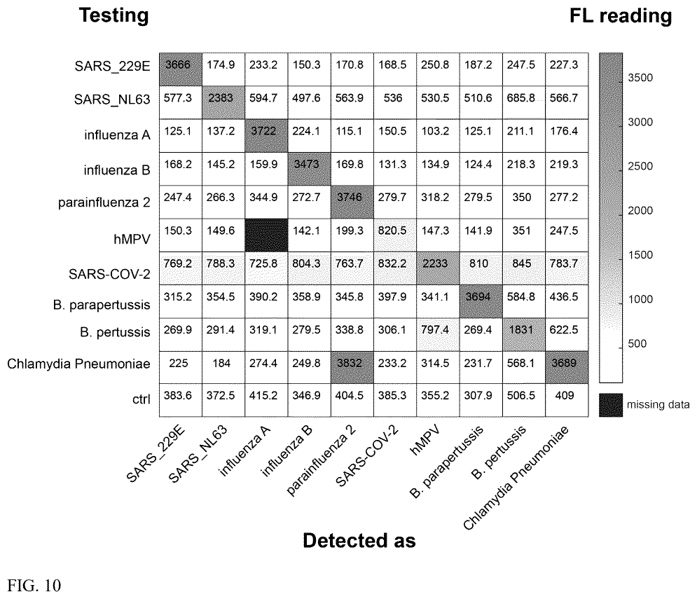

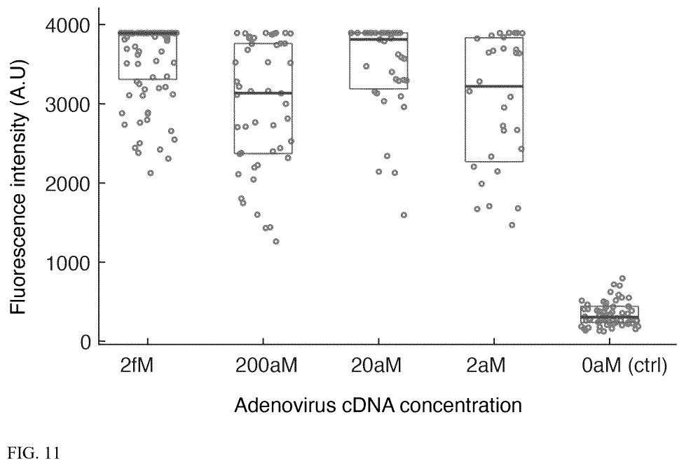

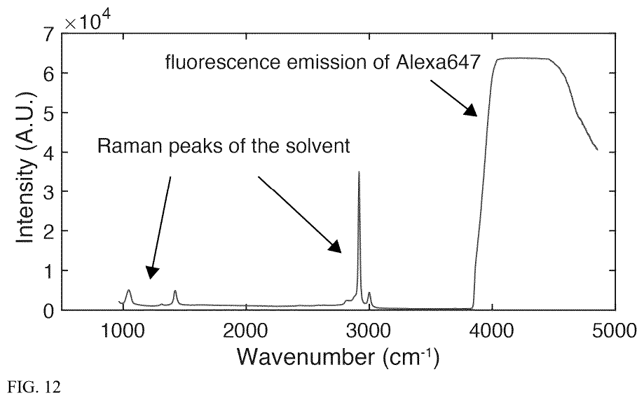

FIG. 10 shows a data matrix summarizes validation results for upper respiratory infection panel, tested with inactivated real viruses and bacteria.

Isotopically-labeled compounds can generally be prepared by conventional techniques known to those skilled in the art using appropriate isotopically-labeled reagents in place of the non-labeled reagents employed.

The term “alkynyl” herein refers to a hydrocarbon radical straight or branched, containing at least 1 carbon-to-carbon triple bond—an “alkyne”—and up to the maximum possible number of non-aromatic carbon-carbon triple bonds may be present. Thus, C2-Cn alkynyl is defined to include groups having 1, 2 . . . , n−1 or n carbons. For example, “C2-C6 alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated. An embodiment can be a C2-Cn alkynyl. An embodiment can be C2-C12 alkynyl or C3-C8 alkynyl.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

1. A method for detecting the presence of one or more different analytes present in a sample comprising:
(a) contacting the sample with a plurality of bead types, each bead type comprising:
(1) one, or more, types of Raman-active small molecule(s), each at a predefined concentration in the bead, and
(2) a binding molecule, affixed thereon, specific for one of each of said one or more different analytes;
wherein each bead type differs from the remaining bead types of the plurality in (i) concentration of Raman-active small molecule(s), (ii) spectrum or spectra of Raman-active small molecule(s), or (ii) both concentration and spectrum/spectra pattern of Raman-active small molecule(s);
(b) removing unbound analytes or unbound beads; and
(c) determining, with Stimulated Raman Scattering (SRS) or Spontaneous Raman Scattering, which bead type(s) having an analyte bound thereto are present after step (b) so as to thereby determine which type(s) of analyte are bound,
thereby detecting which of one or more different analytes are present in the sample.

2. The method of claim 1, wherein each bead type comprises a polystyrene particle.

3. The method of claim 1, wherein all bead types are of the same size, or are all of about the same size.

4. The method of claim 1, wherein each bead type of the plurality has an average diameter of at least 1.0 μm.

5. The method of claim 1, wherein each bead type has peak Raman shift at a predetermined stimulation wavelength of at least 10 cm⁻¹ less, or 10 cm⁻¹ more, than the peak Raman shift of all the other bead types in the plurality.

6. The method of claim 1, wherein the Raman-active small molecules are alkyne-containing and/or do not exceed a molecular weight of 350 g/mol.

7. The method of claim 1, wherein the Raman-active small molecules comprise one or more of the following, wherein “*” adjacent to an alkyne carbon atom indicates presence of a ¹³C isotope:

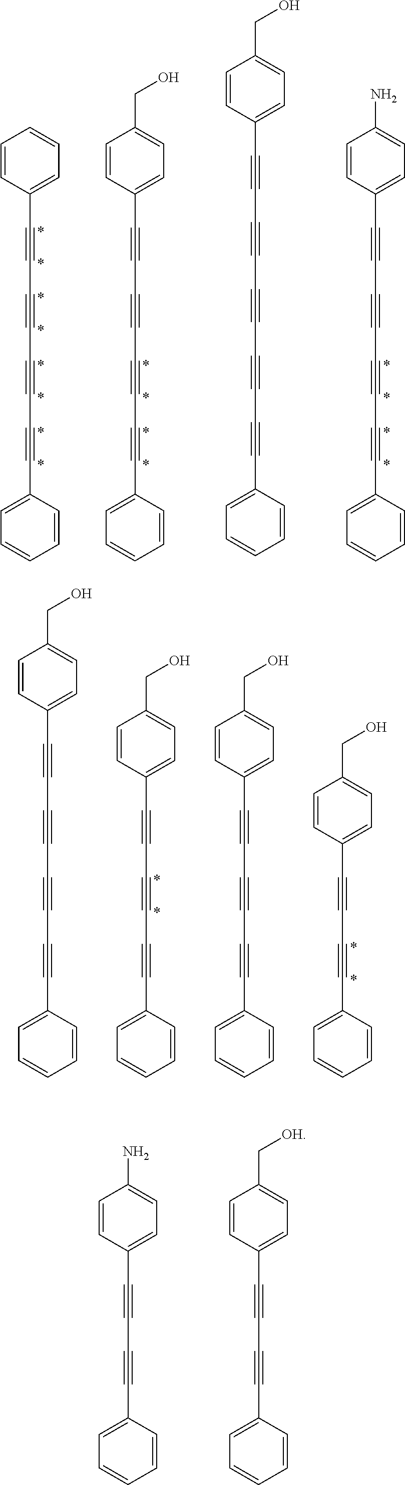

8. The method of claim 1, wherein the Raman-active small molecules comprise one or more of the following, wherein “*” adjacent to an alkyne carbon atom indicates presence of a ¹³C isotope:

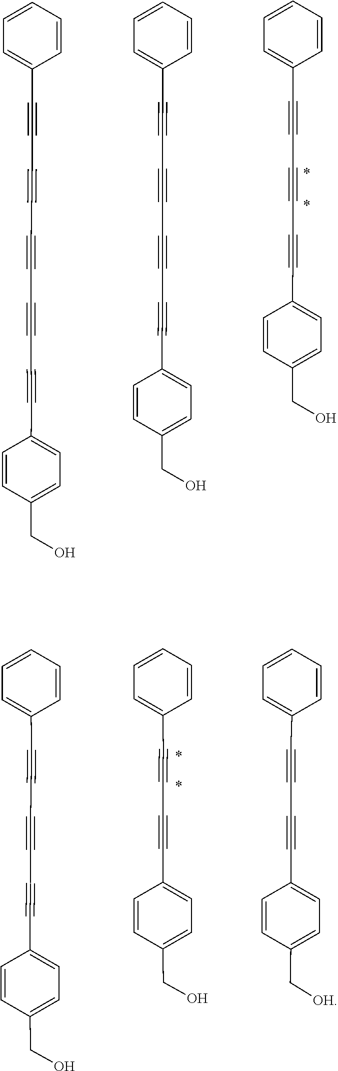 [SMILES: OCc1ccc(C#CC#CC#CC#CC#Cc2ccccc2)cc1 ; OCc1ccc(C#CC#CC#CC#Cc2ccccc2)cc1 ; OCc1ccc(C#CC#CC#Cc2ccccc2)cc1 ; OCc1ccc(C#CC#CC#Cc2ccccc2)cc1 ; OCc1ccc(C#CC#Cc2ccccc2)cc1 ; OCc1ccc(C#CC#Cc2ccccc2)cc1]

9. The method of claim 1, wherein the one or more different analytes are nucleic acids and the binding molecule specific for one of each of said one or more nucleic acids to be detected comprises a complementary nucleic acid capable of hybridizing with the analyte nucleic acid.

10. The method of claim 9, further comprising performing one or more cycles of polymerase chain reaction (PCR) on the analyte nucleic acids of the sample prior to step (a) with one or more primer sequence pairs comprising a forward primer and a reverse primer, the sequence of each of which primers is adjacent to, or flanks, a sequence of the target nucleic acid to which the hybridizing nucleic acid hybridizes.

11. The method of claim 10, further comprising denaturing double-stranded amplicons resulting from the PCR into single-stranded nucleic acids.

12. The method of claim 10, wherein the forward and reverse primers are ended with a repeating 5′ phosphorothioate and the method further comprises contacting the PCR products with a CRISPR cas9 nuclease so as to thereby cleave off 5′ phosphorothioate, thus permitting digestion by lambda-exonuclease so as to form single-stranded DNA.

13. The method of claim 1, wherein the SRS is performed using a 532 nm laser.

14. The method of claim 13, wherein the fluorophore is a far-red fluorophore.

15. The method of claim 14, wherein the fluorophore has an emission maxima greater than 660 nm.

16. The method of claim 15, wherein the fluorophore is excited with a 660 nm laser.

17. A system for Raman spectroscopy and fluorescence spectroscopy of a sample of microbeads doped with Raman-active-small-molecules (RASMs), comprising:
a spectrometer;
a light source comprising a first laser and a second laser, the first laser configured to excite Raman scattering from the sample with signature peaks at a first wavelength and the second laser configured to generate an emission spectrum from the sample with wavelengths higher than the first wavelength;
a sample holder through which the sample is passed during spectroscopy of the sample;
telescopic lenses configured to collimate and expand laser beams emitted by the first and second lasers;
reflective lenses for directing the laser beams to the sample holder;
an objective lens configured to focus an emitted Raman signal and an emitted fluorescence frequency spectrum from the sample resulting from illumination of the sample in the sample holder by the laser beams;
a dichroic beam splitter configured to direct the emitted Raman signal and the emitted fluorescence frequency spectrum to the objective lens;
a pin hole through which the focused Raman signal and the fluorescence frequency spectrum are passed; and
relay lenses positioned after the pinhole for directing the Raman signal and the fluorescence frequency spectrum to the spectrometer, the spectrometer comprising a diffraction grating that filters the Raman signal from the fluorescence frequency spectrum so that both the Raman signal and the fluorescence frequency spectrum are detected by the spectrometer.

18. The system of claim 17, wherein the sample holder comprises a microfluidic device.

19. The system of claim 17, further comprising an inverted microscope that comprises the dichroic beam splitter and the objective lens.

20. The system of claim 17, further comprising relay lenses between the pinhole and the spectrometer.
